(19) United States
(12) Patent Application Publication
(10) Pub. No.: US 2025/0331741 A1
(43) Pub. Date: Oct. 30, 2025

(54) TEMPORALLY ALIGNING PHYSIOLOGICAL PARAMETER MEASUREMENTS

(71) Applicant: Medtronic, Inc., Minneapolis, MN (US)

(72) Inventor: Tyler W. Stigen, Brooklyn Park, MN (US)

(21) Appl. No.: 19/184,100

(22) Filed: Apr. 21, 2025

Related U.S. Application Data
(60) Provisional application No. 63/638,838, filed on Apr. 25, 2024.

(51) Int. Cl. A61B 5/145; A61B 5/00

(52) U.S. Cl. CPC A61B 5/14532; A61B 5/14503; A61B 5/4839; A61B 5/7282

(57) ABSTRACT

A system includes an implantable medical device configured to measure blood-glucose concentration based on cardiac activity. The system further includes processing circuitry configured to generate, based on the plurality of periods, a plurality of waveforms representative of the blood-glucose concentration. The processing circuitry is further configured to identify at least one clinically significant feature that is present in each waveform. The processing circuitry is further configured to modify one or more of the plurality of waveforms such that the at least one feature is temporally aligned across the plurality of waveforms.

### RELATED APPLICATIONS

This application claims the benefit of U.S. Provisional Application Ser. No. 63/638,838, filed Apr. 25, 2024, the entire contents of each of which are incorporated herein by reference.

### TECHNICAL FIELD

The disclosure relates to measuring and analyzing physiological parameters, particularly blood glucose.

### BACKGROUND

People with diabetes may use a medical device, such as a continuous glucose monitor (CGM), to frequently monitor blood glucose levels. In some examples, the medical device may be configured to determine trends and patterns based on the glucose data. In this way, glucose monitoring may help people with diabetes make more informed decisions about their diet, physical activity, insulin dosing, and other aspects of diabetes management, potentially reducing the risk of complications associated with high or low glucose levels.

### SUMMARY

In general, the disclosure describes a system configured to temporally align glucose data from different days or other different periods in order to better identify trends and patterns relevant to diabetes management. For example, the system may use signal processing techniques to realign or remap glucose data from different periods to better identify long-term trends and patterns without losing critical information (that otherwise can occur due to, e.g., temporal averaging). In turn, the system may more effectively identify clinically significant similarities and differences in the periodic glucose data despite idiosyncratic variations in glucose levels that can occur for any number of reasons (e.g., eating or exercising at a different time of day). As a result, the system may more accurately monitor and assess diabetes, which may lead to improved diabetes management (e.g., via the delivery of treatment, the issuance of alerts, etc.).

In one example, a system includes an implantable medical device configured to measure, for a plurality of periods, blood-glucose concentration based on cardiac activity; and processing circuitry configured to: generate, based on the blood-glucose concentration measured during each of the plurality of periods, a corresponding plurality of waveforms representative of the blood-glucose concentration; identify at least one feature that is present in each waveform of the plurality of waveforms, wherein the at least one feature is clinically significant; and modify one or more of the plurality of waveforms such that the at least one feature is temporally aligned across the plurality of waveforms.

In one example, an implantable medical device includes sensing circuitry configured to measure, for a plurality of periods, blood-glucose concentration based on cardiac activity; and processing circuitry configured to: generate, based on the blood-glucose concentration measured during each of the plurality of periods, a corresponding plurality of waveforms representative of the blood-glucose concentration; identify at least one feature that is present in each waveform of the plurality of waveforms, wherein the at least one feature is clinically significant; and modify one or more of the plurality of waveforms such that the at least one feature is temporally aligned across the plurality of waveforms.

In one example, a method includes measuring, by sensing circuitry and for a plurality of periods, blood-glucose concentration based on cardiac activity; and generating, by processing circuitry and based on the blood-glucose concentration measured during each of the plurality of periods, a corresponding plurality of waveforms representative of the blood-glucose concentration; identifying, by the processing circuitry, at least one feature that is present in each waveform of the plurality of waveforms, wherein the at least one feature is clinically significant; and modifying, by the processing circuitry, one or more of the plurality of waveforms such that the at least one feature is temporally aligned across the plurality of waveforms.

The details of one or more examples of the disclosure are set forth in the accompanying drawings and the description below. Other features, objects, and advantages of the disclosure will be apparent from the description and drawings, and from the claims.

### BRIEF DESCRIPTION OF DRAWINGS

FIG. 1 is a conceptual diagram illustrating an example of a medical device system in accordance with techniques of this disclosure.

FIGS. 2A-2B are conceptual diagrams illustrating example glucose signals measured during different periods by an implantable medical device in accordance with techniques of this disclosure.

FIG. 3 is a block diagram illustrating an example configuration of an implantable medical device in accordance with techniques of this disclosure.

FIGS. 4A-4B are conceptual diagrams illustrating signal processing techniques.

FIG. 5 is a functional block diagram illustrating an example configuration of an external device in accordance with techniques of this disclosure.

FIG. 6 is a block diagram illustrating an example system that includes an access point, a network, external computing devices, such as a server, and one or more other computing devices, which may be coupled to an IMD and external device in accordance with techniques of this disclosure.

FIG. 7 is a flow diagram illustrating an example operation of the techniques of this disclosure.

### DETAILED DESCRIPTION

Diabetes is a medical condition characterized by elevated levels of glucose (sugar) in the blood. Elevated glucose levels can cause various health complications if not managed properly. In general, medical devices, such as glucose monitors, may aid diabetes management by measuring glucose levels. For example, a continuous glucose monitor (CGM) may monitor a patient's glucose levels in real-time and help the patient track trends, patterns, etc., in the patient's glucose levels. Such analysis can lead to better glycemic control, reducing the risk of complications associated with diabetes.

In general, a person's glucose levels can fluctuate throughout the day due to a variety of factors, including diet, physical activity, hormones, medications, and underlying health conditions. Certain events like eating and exercising may contribute to the clinically significant content of a patient's glucose data. However, while these events may be regular, the events may not be perfectly periodic (e.g., the events may occur at slightly different times of the day). As a result, if a medical device system performs temporal averaging on the patient's glucose data across multiple days or other periods (e.g., to remove noise and/or identify a trend or pattern), important transient features of the glucose signal may be smoothed out or obscured, potentially leading to the loss of critical information or misinterpretation of the glucose signal.

In accordance with techniques of this disclosure, a medical device system may use signal processing techniques to realign or remap glucose data to better identify long-term trends and patterns without losing critical information (that otherwise can occur due to, e.g., temporal averaging). In turn, the system may more effectively identify clinically significant similarities and differences in the glucose data despite idiosyncratic variations in glucose levels that can occur for any number of reasons (e.g., eating or exercising at a different time). As a result, the system may more accurately monitor and assess diabetes, which may lead to improved diabetes management (e.g., via the delivery of treatment, the issuance of alerts, etc.).

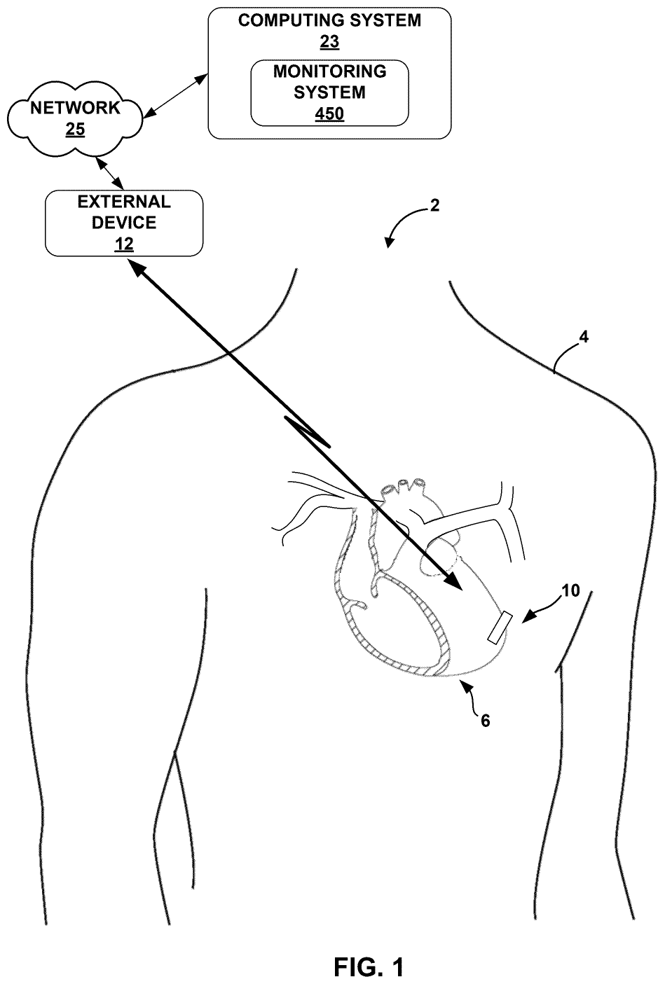

FIG. 1 is a conceptual diagram illustrating an example of a medical device system 2 (“system 2”) in accordance with techniques of this disclosure. In some examples, IMD 10 is implanted outside of a thoracic cavity of patient 4 (e.g., subcutaneously in the pectoral location illustrated in FIG. 1). IMD 10 may be positioned near the sternum near or just below the level of the heart of patient 4, e.g., at least partially within the cardiac silhouette. IMD 10 may include a plurality of electrodes configured to sense electrical signals (e.g., an electrocardiogram (ECG or another cardiac electrogram (EGM)). In some examples, IMD 10 may be an insertable cardiac monitor (ICM) configured to continuously monitor the heart's electrical activity.

External device 12 is a computing device configured for wireless communication with IMD 10. External device 12 may be, as examples, a mobile telephone or other computing device of patient 4 or another user, or a computing device detected to communication with IMD 10. External device 12 may be configured to communicate with a computing system 23 via a network 25. In some examples, external device 12 may provide a user interface and allow a user to interact with IMD 10. Computing system 23 may comprise computing devices configured to allow a user to interact with IMD 10, or data collected from IMD 10, via network 25.

In some examples, computing system 23 includes one or more handheld computing devices, computer workstations, servers or other networked computing devices. In some examples, computing system 23 may include one or more devices, including processing circuitry and storage devices, that implement a monitoring system 450. Computing system 23, network 25, and monitoring system 450 may be implemented by the Medtronic Carelink™ Network or other patient monitoring system, in some examples.

IMD 10 may be configured to continuously monitor the glucose levels of patient 4. For example, IMD 10 may measure blood-glucose concentration based on cardiac activity (e.g., represented by an ECG). IMD 10 may collect data for an extended period of time (including a plurality of periods, such as multiple days, weeks, etc.) such that it may be desirable, if not necessary, to condense the data into meaningful summaries. For example, system 2 may perform temporal averaging to the glucose signal to reduce noise, smooth out irregularities or fluctuations, identify trends, compress data, etc. However, because clinically significant events (e.g., eating, exercising, etc.) may not be perfectly periodic, temporal averaging may inadvertently obscure or distort critical information within the glucose signal. Thus, temporal averaging may not be an effective technique for processing a patient's glucose data.

In accordance with techniques of this disclosure, system 2 (e.g., processing circuitry of one or more devices of system 2) may use signal processing techniques to realign or remap glucose data to better identify long-term trends and patterns without losing critical information. For example, system 2 may stretch or compress a glucose signal in the time dimension to allow for the comparison of glucose signals that have different temporal structures. By manipulating the temporal distortions and aligning important features (that are associated with clinically significant events like eating, exercising, etc.), system 2 may better extract meaningful information from the glucose data, which system 2 may in turn use to aid treatment and other aspects of diabetes management.

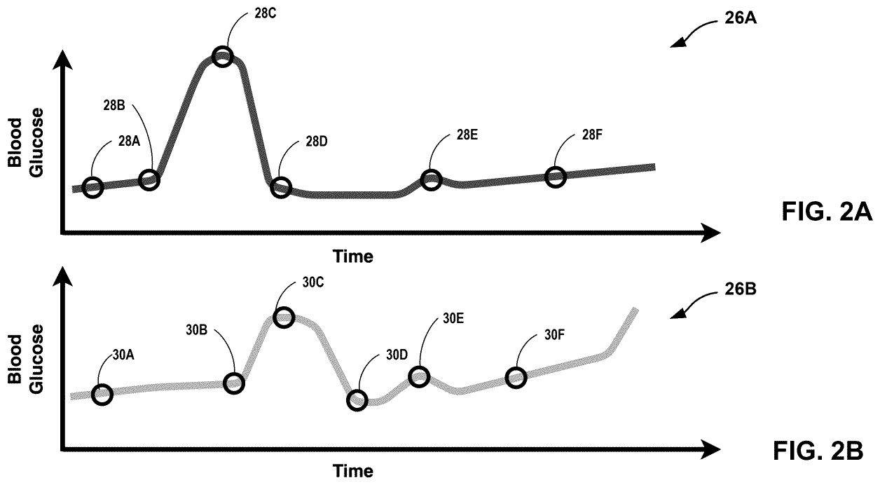

System 2 may generate, based on the blood-glucose concentration measured during each of a plurality of periods, a corresponding plurality of waveforms representative of the blood-glucose concentration. For example, FIGS. 2A-2B are conceptual diagrams of glucose signals measured by IMD 10. In particular, FIG. 2A illustrates an example glucose waveform 26A, and FIG. 2B illustrates an example glucose waveform 26B (collectively, “waveforms 26”). Waveforms 26 may represent a time series, or a sequence of chronological data points collected or recorded by sensors of IMD 10 and/or determined by processing circuitry of IMD 10. For example, electrodes of IMD 10 may sense an ECG of patient 4 and processing circuitry of IMD 10 may determine a time series of blood-glucose concentration values based on the ECG, e.g., based on one or more periodically occurring morphological features of the ECG. As such, waveforms 26 may represent the blood-glucose concentrations of patient 4 over a period (or periods) of time.

In some examples, IMD 10 may collect the measurements at a regular frequency (e.g., a sampling rate) and associate each data point with a timestamp that allows for the temporal ordering of the data points. In some examples, system 2 may display the data using plots such as line charts or waveform displays to help a clinician or other user to identify trends, patterns, and abnormalities in the data.

Glucose waveform 26A may represent a time series of a patient's glucose levels for a first period of time, such as a first day (e.g., a first 24-hour period). Similarly, glucose waveform 26B may represent a time series of a patient's glucose levels for a second period of time, such as a second day (e.g., a second 24-hour period). Waveforms 26 may be substantially similar to each other in shape. However, due to a variety of factors (e.g., the exact time patient 4 eats, exercises, sleeps, etc.), clinically significant features of waveforms 26 may not be temporally aligned.

For example, as shown in FIGS. 2A-2B, system 2 may identify features 28A-28F (collectively, “features 28”) of glucose waveform 26A and features 30A-30F (collectively, “features 30”) of glucose waveform 26B. Features 28 and features 30 somewhat correspond to each other, but features 28 and features 30 are clearly not synchronized with respect to the time they occur during their respective periods. Furthermore, if waveforms 26 were temporally averaged, e.g., to reduce of the volume of data for review by a user or algorithm, and/or to reduce the prominence of noise, features 28 and features 30 would be obscured, potentially frustrating analysis of features 28 and features 30. For example, temporally averaging waveforms 26 may result in the loss of one or more of features 28 and features 30 due to temporal averaging smoothing out abrupt changes like sudden events, spikes, or fluctuations in the signal. Accordingly, temporal averaging may misrepresent waveforms 26, which may lead to inaccurate analysis of the data.

In accordance with techniques of this disclosure, system 2 may modify at least one of waveforms 26 to temporally align features 28 and features 30. Temporal alignment may refer to temporal realignment, non-linear temporal realignment, non-transpositional realignment, etc. In some examples, system 2 may perform dynamic time warping (DTW) or another suitable methodology (e.g., Needlemen-Wunsch) to stretch or compress at least one of waveforms 26, thereby realigning or remapping features 28 and/or features 30. System 2 may use DTW to maximize the similarity between waveforms 26, thereby facilitating comparison between waveforms 26 that can lead to the identification of trends and patterns in waveforms 26.

DTW may include determining an optimal warping path through waveforms 26, which may involve local time shifts, expansions, and/or compressions to align features 28 and 30. In some examples, system 2 (e.g., processing circuitry of one or more devices of system 2) may determine the optimal warping path by computing a cost matrix. Each element in the cost matrix may represent the cost of aligning a data point from one waveform (e.g., waveform 26A) with a data point from the other waveform (e.g., waveform 26B). The optimal warping path may represent the alignment that minimizes the total cost of matching waveforms 26, in this way synchronizing waveforms 26, particularly features 28 and 30.

By aligning or otherwise synchronizing waveforms 26 as described above, system 2 may summarize the glucose data while reducing the risk of data being lost or obscured. For example, DTW may retain the essential properties (e.g., magnitude, shape, etc.) of features 28 and features 30 instead of diluting or dampening features 28 and features 30 as a result of temporal averaging. That said, it should be noted that, in some examples, system 2 may perform temporal averaging after performing DTW (or another suitable signal processing technique) to summarize the data.

In any case, after aligning features 28 and features 30, system 2 may process features 28 and features 30 to facilitate diabetes management. For example, features 28 and features 30 may represent or otherwise be associated with baseline glucose levels, peak glucose levels, trough glucose levels, rate of glucose change, glucose variability, hypoglycemic events, hyperglycemic events, etc. In some examples, system 2 may identify events or other contextual information associated with features 28 and features 30 based on correlations, patterns, etc. Example correlations may include a large, sudden increase in a patient's glucose levels being associated with eating, and a large, sudden decrease in a patient's glucose levels being associated with exercising.

System 2 may determine a condition of patient 4 based on features 28 and features 30 and perform a corresponding action. For example, system 2 may control, administer, adjust, or otherwise regulate therapy delivery (e.g., insulin administration) to manage glucose levels before, during, and/or after eating, exercising, etc. System 2 may also provide real-time or predictive alerts to help patient 4 (and other users, such as a clinician) monitor glucose level changes. For example, the alerts may indicate the patient's glucose levels, glucose level changes, possible causes for the glucose level changes, trends in the glucose levels, etc. Access to this information may help patient 4 avoid dangerous situations, such as hypoglycemia and hyperglycemia, and inform patient 4 of trends (e.g., the direction and speed of glucose level changes) that may indicate the need for adjustments in medication, diet, and/or physical activity.

Thus, by using DTW (or another suitable signal processing technique), system 2 may compare waveforms 26 that have varying temporal structures. Temporally aligning features 28 and 30 may enable analysis and extraction of meaningful information from waveforms 26 that otherwise could be obscured by other signal processing methods (e.g., temporal averaging). For example, applying DTW may improve summarization of data, potentially enabling more accurate analysis of key characteristics, trends, patterns, etc.

Although primarily described herein as using DTW, the techniques may use any suitable signal processing technique to align features 28 and features 30 of waveforms 26. For example, system 2 may additionally or alternatively use the earth mover's distance method. Furthermore, the techniques may be used with any number of waveforms greater than one (e.g., three waveforms, four waveforms, five waveforms, etc.). Furthermore the techniques may be used with physiological parameters other than glucose levels. Thus, examples other than those explicitly described herein are also contemplated by this disclosure. Furthermore, although primarily described herein as using daily periods, the periods may be of any length (e.g., may capture weekly or monthly events). In some examples, system 2 may combine periods to form longer periods or timeframes. In general, system 2 may apply the techniques to any time period or length of time (e.g., system 2 may analyze glucose excursions over the last few weeks or months).

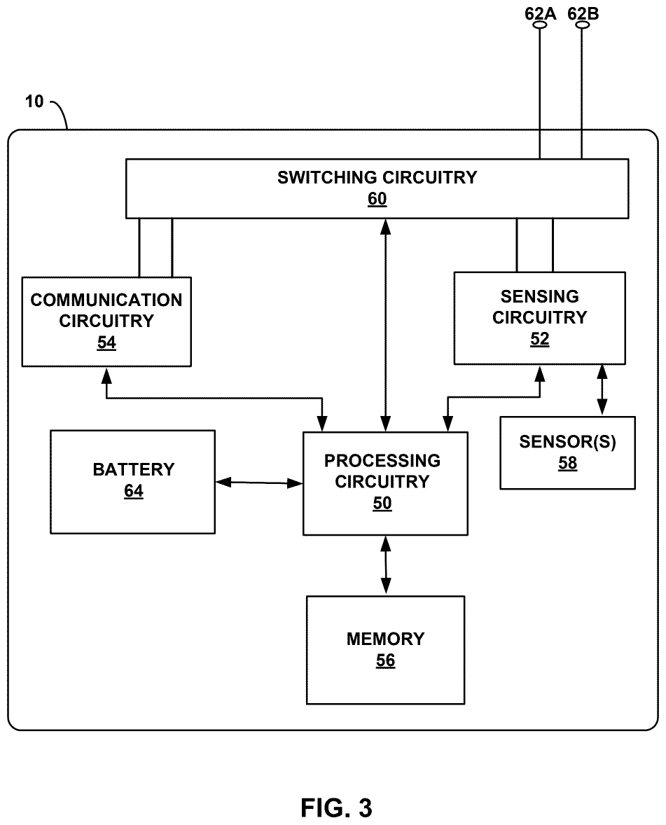

FIG. 3 is a block diagram illustrating an example configuration of IMD 10. In the illustrated example, IMD 10 includes processing circuitry 50 sensing circuitry 52, communication circuitry 54, memory 56, sensors 58, switching circuitry 60, electrodes 62A, 62B (hereinafter “electrodes 62”), and a battery 64.

Processing circuitry 50 may include fixed function circuitry and/or programmable processing circuitry. Processing circuitry 50 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be embodied as software, firmware, hardware or any combination thereof.

In some examples, memory 56 includes computer-readable instructions that, when executed by processing circuitry 50, cause IMD 10 and processing circuitry 50 to perform various functions attributed herein to IMD 10 and processing circuitry 50. Memory 56 may include any volatile, non-volatile, magnetic, optical, or electrical media, such as a random-access memory (RAM), read-only memory (ROM), non-volatile RAM (NVRAM), electrically-erasable programmable ROM (EEPROM), flash memory, or any other digital media.

Sensing circuitry 52 may be selectively coupled to electrodes 62 via switching circuitry 60 as controlled by processing circuitry 50. Sensing circuitry 52 may monitor signals from electrodes 62 in order to monitor electrical activity, such as electrical activity of a heart of patient 4 of FIG. 1. The electrical activity may represent cardiac activity of the heart of patient 4. Processing circuitry 50 may determine blood-glucose concentration based on the cardiac activity of the heart of patient 4. For example, processing circuitry 50 may use relationships between glucose levels and various parameters like heart rate variability (HRV), QT interval, PR interval, the magnitude of R wave and T wave, as well as relative ratios of these different metrics.

In some examples, processing circuitry 50 transmits, via communication circuitry 54, the episode data for patient 4 to an external device, such as external device 12 of FIG. 1. For example, IMD 10 may send digitized cardiac EGM and other episode data to network 25 for processing by monitoring system 450 of FIG. 1.

In some examples, IMD 10 includes one or more sensors 58, such as one or more accelerometers, microphones, optical sensors, and/or pressure sensors. In some examples, sensing circuitry 52 may include one or more filters and amplifiers for filtering and amplifying signals received from one or more of electrodes 62 and/or other sensors 58. In some examples, sensing circuitry 52 and/or processing circuitry 50 may include a rectifier, filter and/or amplifier, a sense amplifier, comparator, and/or analog-to-digital converter. Processing circuitry 50 may determine values of physiological parameters of patient 4 based on signals from sensors 58.

Communication circuitry 54 may include any suitable hardware, firmware, software or any combination thereof for communicating with another device, such as external device 12. Communication circuitry 54 may be configured to communicate using any of a variety of wireless communication schemes, such as Bluetooth® or Bluetooth Low Energy®. Under the control of processing circuitry 50, communication circuitry 54 may receive downlink telemetry from, as well as send uplink telemetry to, external device 12 or another device with the aid of an internal or external antenna. In some examples, processing circuitry 50 may communicate with a networked computing device via an external device (e.g., external device 12) and a computer network, such as the Medtronic CareLink® Network developed by Medtronic, plc, of Dublin, Ireland.

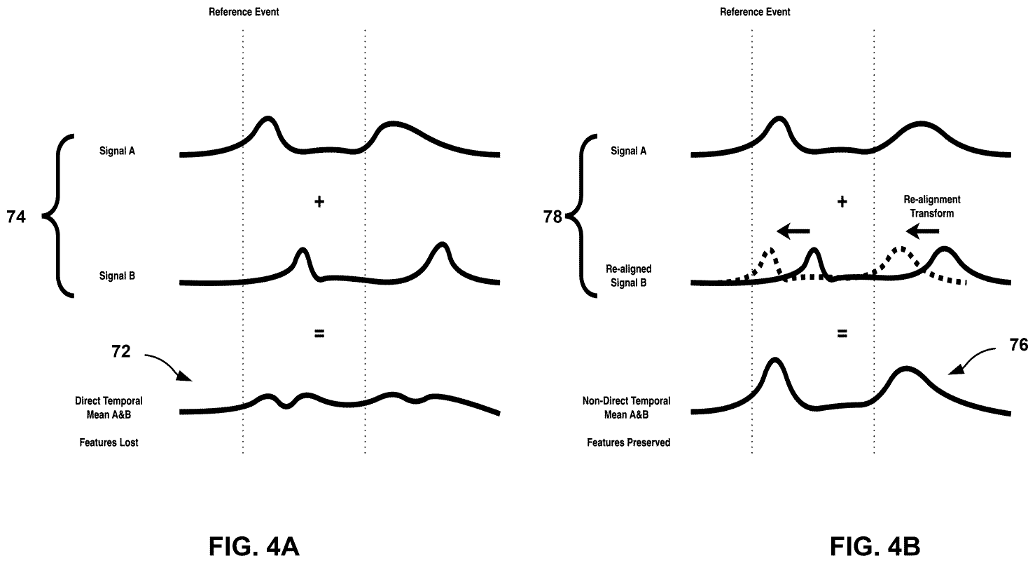

FIGS. 4A-4B are conceptual diagrams illustrating signal processing techniques. FIG. 4A illustrates a waveform 72 resulting from temporal averaging without feature alignment. As shown in FIG. 4A, waveform 72 misrepresents signals 74 because waveform 72 includes six small peaks instead of three large peaks. By contrast, FIG. 4B illustrates a waveform 46 resulting from feature alignment and temporal averaging. System 2 may first use DTW to temporally align features of signals 78 and then use temporal averaging to generate waveform 76. As shown in FIG. 4B, waveform 76 accurately represents signals 78 because, like signals 78, waveform 76 includes three large peaks such that the overall shape of waveform 76 is substantially similar to that of signals 78.

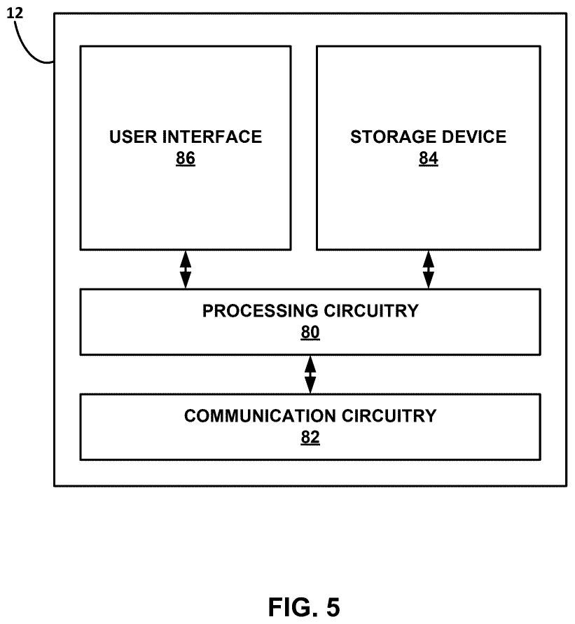

FIG. 5 is a block diagram illustrating an example configuration of components of external device 12. In the example of FIG. 5, external device 12 includes processing circuitry 80, communication circuitry 82, storage device 84, and user interface 86.

Processing circuitry 80 may include one or more processors that are configured to implement functionality and/or process instructions for execution within external device 12. For example, processing circuitry 80 may be capable of processing instructions stored in storage device 84. Processing circuitry 80 may include, for example, microprocessors, DSPs, ASICs, FPGAs, or equivalent discrete or integrated logic circuitry, or a combination of any of the foregoing devices or circuitry. Accordingly, processing circuitry 80 may include any suitable structure, whether in hardware, software, firmware, or any combination thereof, to perform the functions ascribed herein to processing circuitry 80.

Communication circuitry 82 may include any suitable hardware, firmware, software or any combination thereof for communicating with another device, such as IMD 10. Under the control of processing circuitry 80, communication circuitry 82 may receive downlink telemetry from, as well as send uplink telemetry to, IMD 10, or another device. Communication circuitry 82 may be configured to transmit or receive signals via inductive coupling, electromagnetic coupling, Near Field Communication (NFC), Radio Frequency (RF) communication, Bluetooth, WiFi, or other proprietary or non-proprietary wireless communication schemes. Communication circuitry 82 may also be configured to communicate with devices other than IMD 10 via any of a variety of forms of wired and/or wireless communication and/or network protocols.

Storage device 84 may be configured to store information within external device 12 during operation. Storage device 84 may include a computer-readable storage medium or computer-readable storage device. In some examples, storage device 84 includes one or more of a short-term memory or a long-term memory. Storage device 84 may include, for example, RAM, DRAM, SRAM, magnetic discs, optical discs, flash memories, or forms of EPROM or EEPROM. In some examples, storage device 84 is used to store data indicative of instructions for execution by processing circuitry 80. Storage device 84 may be used by software or applications running on external device 12 to temporarily store information during program execution.

Data exchanged between external device 12 and IMD 10 may include operational parameters. External device 12 may transmit data including computer readable instructions which, when implemented by IMD 10, may control IMD 10 to change one or more operational parameters and/or export collected data. For example, processing circuitry 80 may transmit an instruction to IMD 10 which requests IMD 10 to export collected data (e.g., glucose concentration measurements) to external device 12. In turn, external device 12 may receive the collected data from IMD 10 and store the collected data in storage device 84. Processing circuitry 80 may implement any of the techniques described herein to temporally align features of waveforms representing the collected data received from IMD 10, e.g., to temporally align features 28 and features 30 of waveforms 26.

A user, such as a clinician or the patient, may interact with external device 12 through user interface 86. User interface 86 includes a display (not shown), such as a liquid crystal display (LCD) or a light emitting diode (LED) display or other type of screen, with which processing circuitry 80 may present information related to IMD 10, e.g., waveforms representing the collected data received from IMD 10 (including waveforms with temporally aligned features). In addition, user interface 86 may include an input mechanism to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processing circuitry 80 of external device 12 and provide input. In other examples, user interface 86 also includes audio circuitry for providing audible notifications, instructions or other sounds to the user, receiving voice commands from the user, or both.

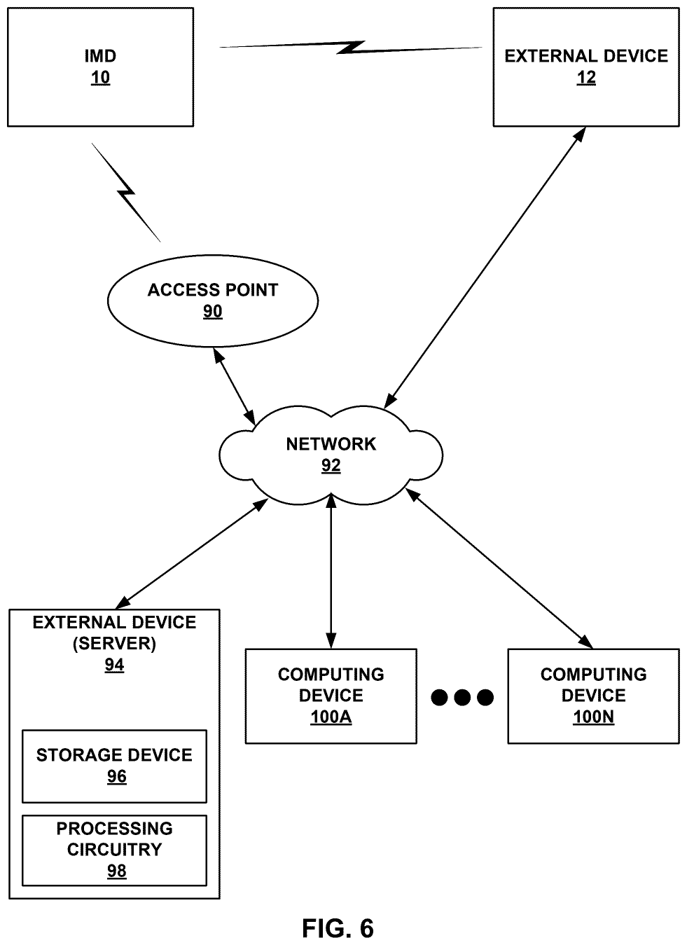

FIG. 6 is a block diagram illustrating an example system that includes an access point 90, a network 92, external computing devices, such as a server 94, and one or more other computing devices 100A-100N (collectively, “computing devices 100”), which may be coupled to IMD 10 and external device 12 via network 92, in accordance with one or more techniques described herein. In this example, IMD 10 may use communication circuitry 54 to communicate with external device 12 via a first wireless connection, and to communicate with an access point 90 via a second wireless connection. In the example of FIG. 6, access point 90, external device 12, server 94, and computing devices 100 are interconnected and may communicate with each other through network 92.

Access point 90 may include a device that connects to network 92 via any of a variety of connections, such as telephone dial-up, digital subscriber line (DSL), or cable modem connections. In other examples, access point 90 may be coupled to network 92 through different forms of connections, including wired or wireless connections. In some examples, access point 90 may be a user device, such as a tablet or smartphone, that may be co-located with the patient. IMD 10 may be configured to transmit data, such as data regarding a physiological parameter of interest like glucose concentrations, to access point 90. Access point 90 may then communicate the retrieved data to server 94 via network 92.

In some cases, server 94 may be configured to provide a secure storage site for data that has been collected from IMD 10 and/or external device 12. In some cases, server 94 may assemble data in web pages or other documents for viewing by trained professionals, such as clinicians, via computing devices 100. One or more aspects of the illustrated system of FIG. 6 may be implemented with general network technology and functionality, which may be similar to that provided by the Medtronic CareLink® Network.

In some examples, one or more of computing devices 100 may be a tablet or other smart device located with a clinician, by which the clinician may program, receive data from, and/or interrogate IMD 10. For example, the clinician may access data collected by IMD 10 through a computing device 100, such as when a patient is in in between clinician visits, to check on a status of a medical condition or the operation of IMD 10. In some examples, the clinician may enter instructions for a medical intervention for the patient into an application executed by computing device 100, such as based on a status of a patient condition determined by IMD 10, external device 12, server 94. or any combination thereof, or based on other patient data known to the clinician. Device 100 then may transmit the instructions for medical intervention to another of computing devices 100 located with the patient or a caregiver of the patient. For example, such instructions for medical intervention may include an instruction to change a drug dosage, timing, or selection, to schedule a visit with the clinician, or to seek medical attention. In further examples, a computing device 100 may generate an alert to the patient based on a status of a medical condition of the patient, which may enable the patient to proactively seek medical attention prior to receiving instructions for a medical intervention. In this manner, the patient may be empowered to take action, as needed, to address the patient's medical status, which may help improve clinical outcomes for the patient.

In the example illustrated by FIG. 6, server 94 includes a storage device 96, e.g., to store data retrieved from IMD 10, and processing circuitry 98. Although not illustrated in FIG. 6, computing devices 100 may similarly include a storage device and processing circuitry. Processing circuitry 98 may include one or more processors that are configured to implement functionality and/or process instructions for execution within server 94. For example, processing circuitry 98 may be capable of processing instructions stored in memory 96. Processing circuitry 98 may include, for example, microprocessors, DSPs, ASICs, FPGAs, or equivalent discrete or integrated logic circuitry, or a combination of any of the foregoing devices or circuitry. Accordingly, processing circuitry 98 may include any suitable structure, whether in hardware, software, firmware, or any combination thereof, to perform the functions ascribed herein to processing circuitry 98. Processing circuitry 98 of server 94 and/or the processing circuity of computing devices 100 may implement any of the techniques described herein to temporally align features of waveforms representing data received from IMD 10, such as features 28 and features 30 of waveforms 26.

Storage device 96 may include a computer-readable storage medium or computer-readable storage device. In some examples, memory 96 includes one or more of a short-term memory or a long-term memory. Storage device 96 may include, for example, RAM, DRAM, SRAM, magnetic discs, optical discs, flash memories, or forms of EPROM or EEPROM. In some examples, storage device 96 is used to store data indicative of instructions for execution by processing circuitry 98.

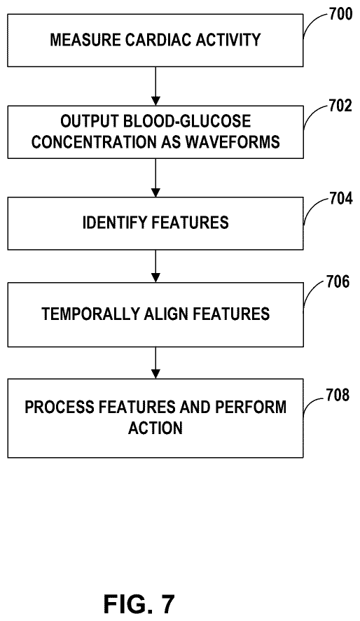

FIG. 7 is a flow diagram illustrating an example operation of system 2. In some examples, electrodes of IMD 10 may measure cardiac activity of patient 4 (e.g., collect ECGs for a plurality of periods) and determine blood-glucose concentration based on the cardiac activity (700). IMD 10 may collect the measurements at a regular frequency (e.g., a sampling rate) and associate each data point with a timestamp that allows for the temporal ordering of the data points. System 2 may output the blood-glucose concentrations as waveforms 26 (e.g., as time series of concentration values) (702).

System 2 may identify clinically significant features in waveforms 26 (704). For example, system 2 may identify features 28 in waveform 26A and features 30 in waveform 26B. Features 28 and features 30 may represent or otherwise be associated with baseline glucose levels, peak glucose levels, trough glucose levels, rate of glucose change, glucose variability, hypoglycemic events, hyperglycemic events, etc.

System 2 may modify waveforms 26 to temporally align features of waveforms 26 (706). For example, system 2 may perform DTW to stretch or compress at least one of waveforms 26, thereby realigning or remapping features 28 and/or features 30. System 2 may use DTW to maximize the similarity between waveforms 26, thereby facilitating comparison between waveforms 26 that can lead to the identification of trends and patterns in waveforms 26. In some examples, system 2 may use earth mover's distance or another similar technique.

After aligning features 28 and features 30, system 2 may process features 28 and features 30 to facilitate diabetes management (708). For example, system 2 may adjust therapy delivery (e.g., insulin administration) to manage glucose levels before, during, and/or after eating, exercising, etc. System 2 may also provide real-time or predictive alerts to help patient 4 (and other users, such as a clinician) monitor glucose level changes. Access to this information may help patient 4 avoid dangerous situations, such as hypoglycemia and hyperglycemia, and inform patient 4 of trends (e.g., the direction and speed of glucose level changes) that may indicate the need for adjustments in medication, diet, and/or physical activity.

This disclosure includes various examples, such as the following examples.

Example 1: A system includes an implantable medical device configured to measure, for a plurality of periods, blood-glucose concentration based on cardiac activity; and processing circuitry configured to: generate, based on the blood-glucose concentration measured during each of the plurality of periods, a corresponding plurality of waveforms representative of the blood-glucose concentration; identify at least one feature that is present in each waveform of the plurality of waveforms, wherein the at least one feature is clinically significant; and modify one or more of the plurality of waveforms such that the at least one feature is temporally aligned across the plurality of waveforms.

Example 2: The system of example 1, wherein the processing circuitry is configured to modify the one or more of the plurality of waveforms by performing at least one of dynamic time warping or Needlemen-Wunsch.

Example 3: The system of example 1 or 2, wherein the processing circuitry is further configured to temporally average the plurality of waveforms subsequent to modifying the one or more of the plurality of waveforms.

Example 4: The system of any of examples 1 to 3, wherein the at least one feature is associated with at least one of a baseline glucose level, a peak glucose level, a trough glucose level, a rate of glucose change, a glucose variability, a hypoglycemic event, or a hyperglycemic events.

Example 5: The system of any of examples 1 to 4, wherein the processing circuitry is further configured to analyze the at least one feature subsequent to temporally aligning the at least one feature across the plurality of waveforms.

Example 6: The system of example 5, wherein the processing circuitry is further configured to control therapy delivery based on the at least one feature.

Example 7: The system of example 6, wherein the processing circuitry is configured to control therapy delivery by controlling delivery of insulin at least one of before or after eating.

Example 8: The system of example 6 or 7, wherein the processing circuitry is configured to control therapy delivery by controlling insulin at least one of before or after exercising.

Example 9: The system of any of examples 5 to 8, wherein the processing circuitry is further configured to provide an alert indicating at least one of a glucose level, a glucose level change, a possible cause for the glucose level change, or a trend in the glucose level.

Example 10: The system of any of examples 1 to 9, wherein each period of the plurality of periods is a day.

Example 11: The system of any of examples 1 to 10, wherein the implantable medical device is an insertable cardiac monitor.

Example 12: An implantable medical device includes sensing circuitry configured to measure, for a plurality of periods, blood-glucose concentration based on cardiac activity; and processing circuitry configured to: generate, based on the blood-glucose concentration measured during each of the plurality of periods, a corresponding plurality of waveforms representative of the blood-glucose concentration; identify at least one feature that is present in each waveform of the plurality of waveforms, wherein the at least one feature is clinically significant; and modify one or more of the plurality of waveforms such that the at least one feature is temporally aligned across the plurality of waveforms.

Example 13: The implantable medical device of example 12, wherein the processing circuitry is configured to modify the one or more of the plurality of waveforms by performing at least one of dynamic time warping or Needlemen-Wunsch.

Example 14: The implantable medical device of example 12 or 13, wherein the processing circuitry is further configured to temporally average the plurality of waveforms subsequent to modifying the one or more of the plurality of waveforms.

Example 15: The implantable medical device of any of examples 12 to 14, wherein the at least one feature is associated with at least one of a baseline glucose level, a peak glucose level, a trough glucose level, a rate of glucose change, a glucose variability, a hypoglycemic event, or a hyperglycemic events.

Example 16: The implantable medical device of any of examples 12 to 15, wherein the processing circuitry is further configured to analyze the at least one feature subsequent to temporally aligning the at least one feature across the plurality of waveforms.

Example 17: The implantable medical device of example 16, wherein the processing circuitry is further configured to control therapy delivery based on the at least one feature.

Example 18: The implantable medical device of example 17, wherein the processing circuitry is configured to control therapy delivery by controlling delivery of insulin at least one of before or after eating.

Example 19: The implantable medical device of example 17 or 18, wherein the processing circuitry is configured to control therapy delivery by controlling insulin at least one of before or after exercising.

Example 20: The implantable medical device of any of examples 16 to 19, wherein the processing circuitry is further configured to provide an alert indicating at least one of a glucose level, a glucose level change, a possible cause for the glucose level change, or a trend in the glucose level.

Example 21: The implantable medical device of any of examples 12 to 20, wherein each period of the plurality of periods is a day.

Example 22: The implantable medical device of any of examples 12 to 21, wherein the implantable medical device is an insertable cardiac monitor.

Example 23: A method includes measuring, by sensing circuitry and for a plurality of periods, blood-glucose concentration based on cardiac activity; and generating, by processing circuitry and based on the blood-glucose concentration measured during each of the plurality of periods, a corresponding plurality of waveforms representative of the blood-glucose concentration; identifying, by the processing circuitry, at least one feature that is present in each waveform of the plurality of waveforms, wherein the at least one feature is clinically significant; and modifying, by the processing circuitry, one or more of the plurality of waveforms such that the at least one feature is temporally aligned across the plurality of waveforms.

Example 24: The method of example 23, further including modifying, by the processing circuitry, the one or more of the plurality of waveforms by performing at least one of dynamic time warping or Needlemen-Wunsch.

Example 25: The method of example 23 or 24, further including temporally averaging, by the processing circuitry, the plurality of waveforms subsequent to modifying the one or more of the plurality of waveforms.

Example 26: The method of any of examples 23 to 25, wherein the at least one feature is associated with at least one of a baseline glucose level, a peak glucose level, a trough glucose level, a rate of glucose change, a glucose variability, a hypoglycemic event, or a hyperglycemic events.

Example 27: The method of any of examples 12 to 15, further including analyzing, by the processing circuitry, the at least one feature subsequent to temporally aligning the at least one feature across the plurality of waveforms.

Example 28: The method of example 27, further including controlling, by the processing circuitry, therapy delivery based on the at least one feature.

Example 29: The method of example 28, wherein controlling therapy delivery includes controlling, by the processing circuitry, delivery of insulin at least one of before or after eating.

Example 30: The method of example 28 or 29, wherein controlling therapy delivery includes controlling, by the processing circuitry, delivery of insulin at least one of before or after exercising.

Example 31: The method of any of examples 27 to 30, further including providing, by the processing circuitry, an alert indicating at least one of a glucose level, a glucose level change, a possible cause for the glucose level change, or a trend in the glucose level.

Example 32: The method of any of examples 23 to 31, wherein each period of the plurality of periods is a day.

Example 33: The method of any of examples 23 to 32, wherein the implantable medical device is an insertable cardiac monitor.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module, unit, or circuit for purposes of clarity, it should be understood that the techniques of this disclosure may be performed by a combination of units, modules, or circuitry associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processing circuitry” as used herein may refer to any of the foregoing structure or any other physical structure suitable for implementation of the described techniques. Also, the techniques could be fully implemented in one or more circuits or logic elements.

Various examples of the disclosure have been described. These and other examples are within the scope of the following claims.

What is claimed is:

1. A system comprising:
an implantable medical device configured to measure, for a plurality of periods, blood-glucose concentration based on cardiac activity; and
processing circuitry configured to:
generate, based on the blood-glucose concentration measured during each of the plurality of periods, a corresponding plurality of waveforms representative of the blood-glucose concentration;
identify at least one feature that is present in each waveform of the plurality of waveforms, wherein the at least one feature is clinically significant; and
modify one or more of the plurality of waveforms such that the at least one feature is temporally aligned across the plurality of waveforms.

2. The system of claim 1, wherein the processing circuitry is configured to modify the one or more of the plurality of waveforms by performing at least one of dynamic time warping or Needlemen-Wunsch.

3. The system of claim 1, wherein the processing circuitry is further configured to temporally average the plurality of waveforms subsequent to modifying the one or more of the plurality of waveforms.

4. The system of claim 1, wherein the at least one feature is associated with at least one of a baseline glucose level, a peak glucose level, a trough glucose level, a rate of glucose change, a glucose variability, a hypoglycemic event, or a hyperglycemic events.

5. The system of claim 1, wherein the processing circuitry is further configured to analyze the at least one feature subsequent to temporally aligning the at least one feature across the plurality of waveforms.

6. The system of claim 5, wherein the processing circuitry is further configured to control therapy delivery based on the at least one feature.

7. The system of claim 6, wherein the processing circuitry is configured to control therapy delivery by controlling delivery of insulin at least one of before or after eating.

8. The system of claim 6, wherein the processing circuitry is configured to control therapy delivery by controlling insulin at least one of before or after exercising.

9. The system of claim 5, wherein the processing circuitry is further configured to provide an alert indicating at least one of a glucose level, a glucose level change, a possible cause for the glucose level change, or a trend in the glucose level.

10. The system of claim 1, wherein each period of the plurality of periods is a day.

11. The system of claim 1, wherein the implantable medical device is an insertable cardiac monitor.

12. An implantable medical device comprising:
sensing circuitry configured to measure, for a plurality of periods, blood-glucose concentration based on cardiac activity; and
processing circuitry configured to:
generate, based on the blood-glucose concentration measured during each of the plurality of periods, a corresponding plurality of waveforms representative of the blood-glucose concentration;
identify at least one feature that is present in each waveform of the plurality of waveforms, wherein the at least one feature is clinically significant; and
modify one or more of the plurality of waveforms such that the at least one feature is temporally aligned across the plurality of waveforms.

13. The implantable medical device of claim 12, wherein the processing circuitry is configured to modify the one or more of the plurality of waveforms by performing at least one of dynamic time warping or Needlemen-Wunsch.

14. The implantable medical device of claim 12, wherein the processing circuitry is further configured to temporally average the plurality of waveforms subsequent to modifying the one or more of the plurality of waveforms.

15. The implantable medical device of claim 12, wherein the at least one feature is associated with at least one of a baseline glucose level, a peak glucose level, a trough glucose level, a rate of glucose change, a glucose variability, a hypoglycemic event, or a hyperglycemic events.

16. The implantable medical device of claim 12, wherein the processing circuitry is further configured to analyze the at least one feature subsequent to temporally aligning the at least one feature across the plurality of waveforms.

17. The implantable medical device of claim 16, wherein the processing circuitry is further configured to control therapy delivery based on the at least one feature.

18. The implantable medical device of claim 17, wherein the processing circuitry is configured to control therapy delivery by controlling delivery of insulin at least one of before or after eating.

19. The implantable medical device of claim 17, wherein the processing circuitry is configured to control therapy delivery by controlling insulin at least one of before or after exercising.

20. A method comprising:
measuring, by sensing circuitry and for a plurality of periods, blood-glucose concentration based on cardiac activity; and
generating, by processing circuitry and based on the blood-glucose concentration measured during each of the plurality of periods, a corresponding plurality of waveforms representative of the blood-glucose concentration;
identifying, by the processing circuitry, at least one feature that is present in each waveform of the plurality of waveforms, wherein the at least one feature is clinically significant; and
modifying, by the processing circuitry, one or more of the plurality of waveforms such that the at least one feature is temporally aligned across the plurality of waveforms.
